(19) United States
(12) Patent Application Publication
(10) Pub. No.: US 2022/0119523 A1
(43) Pub. Date: Apr. 21, 2022

(54) TREATMENT OF CUTANEOUS T CELL LYMPHOMA WITH TARGETING OF CD47 PATHWAY

(71) Applicant: The Board of Trustees of the Leland Stanford Junior University, Stanford, CA (US)

(72) Inventors: Irving L. Weissman, Stanford, CA (US); Kelly Marie Mckenna, Palo Alto, CA (US); Jens-Peter Volkmer, Menlo Park, CA (US); James Chen, Gold River, CA (US)

(21) Appl. No.: 17/428,079

(22) PCT Filed: Feb. 7, 2020
(86) PCT No.: PCT/US2020/017177

§ 371 Date: Aug. 3, 2021

Related U.S. Application Data
(60) Provisional application No. 62/802,819, filed on Feb. 8, 2019.

(51) Int. Cl. C07K 16/28; A61K 38/21; A61K 39/395; A61K 31/635; A61K 31/706; A61K 31/7068; A61P 35/00

(52) U.S. Cl. CPC C07K 16/2803; A61K 38/212; A61K 39/3955; A61P 35/00; A61K 31/706; A61K 31/7068; A61K 31/635

(57) ABSTRACT

Methods are provided for treatment of cutaneous T cell lymphoma with an effective dose of an anti-CD47 agent, optionally combined an additional anti-cancer agent.

### CROSS REFERENCE

This application claims benefit of U.S. Provisional Patent Application No. 62/802,819, filed Feb. 8, 2019, which applications are incorporated herein by reference in their entirety.

The immune system's natural capacity to detect and destroy abnormal cells may prevent the development of many cancers. However, cancer cells are sometimes able to avoid detection and destruction by the immune system. Cancer cells can reduce the expression of tumor antigens on their surface, making it harder for the immune system to detect them; express proteins on their surface that induce immune cell inactivation; and/or induce cells in the microenvironment to release substances that suppress immune responses and promote tumor cell proliferation and survival.

Cancer immunotherapies have been developed to enhance immune responses against tumors, by stimulating specific components of the immune system; or by counteracting signals produced by cancer cells that suppress immune responses. Forms of immunotherapy include blocking immune checkpoint proteins, providing agonists of immune modulators to enhance responsiveness; and the use of antibodies and other agents targeted to tumor specific antigens.

Other forms of immunotherapy exploit the innate immune system. The cell surface protein CD47 on healthy cells and its engagement of a phagocyte receptor, SIRPα, constitutes a key “don't eat-me” signal that can turn off engulfment mediated by multiple modalities, including apoptotic cell clearance and FcR mediated phagocytosis. Blocking the CD47 mediated engagement of SIRPα on a phagocyte, or the loss of CD47 expression in knockout mice, can cause removal of live cells and non-aged erythrocytes. Alternatively, blocking SIRPα recognition also allows engulfment of targets that are not normally phagocytosed. Anti-CD47 antibody treatment has also been shown to not only enable macrophage phagocytosis of cancer, but can also initiate an anti-tumor cytotoxic T cell immune response.

The development of effective cancer therapy is of great clinical interest, and is addressed herein.

Related publications include U.S. Pat. Nos. 8,562,997; 9,399,682; 9,017,675; 9,382,320; 9,151,760; 8,758,750; 8,361,736; 8,709,429; 9,193,955; and 7,514,229 and International Patent Applications US2016/049016; US2016/030997; US2016/036520; US2015/046976; US2015/044304; US2015/057233; US2015/026491; US2015/019954; US2015/010650; US2014/035167; US2014/018743; US2014/038485; US2013/021937; and US2011/066580, each herein specifically incorporated by reference.

### SUMMARY

Methods are provided for improved treatment of cutaneous T cell lymphomas (CTCL). In such methods, CTCL cells are contacted with an effective dose of an agent that blocks signaling between CD47 and SIRPα, i.e. therapeutic CD47 blockade. The methods of the invention can provide for increased overall survival of the individual being treated, and a significant decrease in tumor volume.

In some embodiments the CTCL is classified as Mycosis fungoides (MF). In some embodiments the CTCL is classified as Sezary Syndrome (SS). An individual can be selected for treatment based on age, e.g. where an individual is selected for treatment that is diagnosed with CTCL, and is up to about 50 years of age, from about 50-70 years of age, greater than about 70 years of age. An individual can be selected based on the stage of the disease and/or the tissue distribution, e.g. where an individual is selected for treatment that is diagnosed with CTCL at Stage IIa, Stage IIb, Stage III, Stage IV, etc. In some embodiments an individual selected for treatment is characterized by blood involvement of the lymphoma. In some embodiments an individual selected for treatment is characterized by node/visceral involvement of the lymphoma. In some embodiments an individual selected for treatment is characterized by lymphoma largely contained to cutaneous involvement.

In some embodiments an anti-CD47 therapy is combined with one or more additional anti-cancer treatments. Where a combination of agents is provided, the agents can be administered concomitantly, i.e. each agent is administered within about 45 days, 30 days, 15 days, 7 days, 3 days, 2 days, 1 day or substantially simultaneously with respect to the other agent(s) in the combination. The agents can be considered to be combined if administration scheduling is such that the both agents are at a therapeutic level in the individual for at least overlapping periods of time. A benefit of the present invention can be the use of lowered doses of one or more of the agents relative to the dose required as a monotherapy; and or a synergistic therapeutic effect. Further, the combination can provide for increased overall survival of the individual that is treated. Administration may be repeated as necessary for depletion of the cancer cell population.

In some embodiments, anti-CD47 therapy is combined with one or more of skin-directed therapies such as PUVA, bexarotene; biologic-response modifiers; tumor-targeted antibodies; chemotherapy; and extracorporeal photophoresis (ECP); etc. ECP has been reported to lead to monocyte activation, including significant changes in gene expression, and dendritic cell differentiation; and can benefit from combination with the phagocytic cell effect of anti-CD47 agents.

In some embodiments, anti-CD47 therapy is combined with one or both of a Bcl-2 inhibitor; and a hypomethylating agent. In some embodiments the Bcl-2 inhibitor is venetoclax. Optionally, cancer cells are tested for expression of Bcl-2 prior to treatment. In some embodiments the hypomethylating agent is azacitidine or gemcitabine.

In some embodiments, anti-CD47 treatment is combined with administration of a retinoid, which can be topically or systemically administered, e.g. bexarotene, etc.

In some embodiments, anti-CD47 treatment is combined with administration of an HDAC inhibitor, including those currently approved for treatment of CTCL, e.g. Vorinostat (suberoylanilide hydroxamic acid, SAHA), romidepsin (depsipeptide), etc.

In some embodiments, anti-CD47 treatment is combined with administration of interferon alpha.

In some embodiments, anti-CD47 treatment is combined with administration of a tumor targeted antibody, e.g. an antibody specific for CD52; CD2, CD4, CD25, CD30, CCR4, etc., including antibodies currently approved for treatment of CTCL, e.g. alemtuzumab, mogamulizumab, brentuximab vedotin, etc., which can be administered systemically or cutaneously. In some embodiments a combination of an antibody specific for CD47 and an antibody specific for CCR4 are administered as a combination therapy.

In some embodiments a primer agent is administered prior to administering a therapeutically effective dose of an anti-CD47 agent to the individual. Suitable primer agents include an erythropoiesis-stimulating agent (ESA), including, for example, Aranesp® (darbepoetin alfa), Epogen®NF/Procrit®NF (epoetin alfa), Omontys® (peginesatide), Procrit®, etc., and/or a sub-therapeutic dose of an anti-CD47 agent. Following administration of the priming agent, and allowing a period of time effective for an increase in reticulocyte production, a therapeutic dose of an anti-CD47 agent is administered. The therapeutic dose can be administered in number of different ways. In some embodiments, two or more therapeutically effective doses are administered after a primer agent is administered. In some embodiments a therapeutically effective dose of an anti-CD47 agent is administered as two or more doses of escalating concentration, in others the doses are equivalent.

An anti-CD47 agent for use in the methods of the invention interferes with binding between CD47 present on the cancer cell and SIRPα present on a phagocytic cell. Suitable anti-CD47 agents include soluble SIRP polypeptides; soluble CD47; anti-CD47 antibodies, anti-SIRPα antibodies, and the like, where the term antibodies encompasses antibody fragments and variants thereof, as known in the art. In some embodiments the anti-CD47 agent is an anti-CD47 antibody. In some embodiments the anti-CD47 antibody is a non-hemolytic antibody, i.e. a therapeutic dose does not cause significant hemolysis in the patient. In some embodiments the antibody comprises a human IgG4 Fc region.

The contacting of a cancer cells may be performed in vivo, e.g. for therapeutic purposes, and in vitro, e.g. for screening assays and the like. CTCL cells are targeted for depletion by contacting the immune cells, including phagocytic cells, in proximity of the tumor cells with a combination of a CD47 blocking agent that is effective to block the interaction between CD47 and SIRPα, optionally in a combination therapy.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. CTCL cell lines were stained with antibodies for markers as indicated, and the analyzed by flow cytometry for the presence of the markers. HH is a mature T cell line derived from peripheral blood of a patient with aggressive cutaneous T cell leukemia/lymphoma. HUT78 is a cutaneous T cell from a Sezary Syndrome patient. It is shown that that both cell lines express high levels of CD47.

FIG. 2A-2B. The percent of phagocytosis (FIG. 2A) and percent of maximum phagocytosis (FIG. 2B) is shown for the CTCL cell lines using macrophage from NOD scid gamma mice (NSG) in the presence of an IgG4 control antibody, or 5F9-IgG4 antibody, which specifically binds to CD47 on the human cancer cells. The cell line SW620 is a colorectal adenocarcinoma cell line provided as a control. The data show that both CTCL cell lines have increased phagocytosis in the presence of the CD47 blocking agent.

FIG. 3A-3B show the results of a phagocytosis assay as performed for FIG. 2, with magrolimab antibody, which specifically binds human CD47, alone or in combination with an antibody that specifically targets human CCR4, e.g. in the presence of an antibody that corresponds to mogamulizumab, a defucosylated anti-CCR4 humanized IgG1 antibody that binds with high affinity to the N-terminal domain of CCR4, but is not internalized and does not exhibit complement-dependent cytotoxic activity or directly induce apoptosis. The data show that anti-CCR4 by itself has little or no effect on phagocytosis, but synergizes with anti-CD47 antibody

FIG. 4A-4B shows the result of tumor xenograft assays of NSG mice engrafted with HH tumor cells, and treated with a PBS negative control or Magrolimab antibody; FIG. 4A tumor volume and FIG. 4B survival. Survival was improved, and tumor volume was reduced by the treatment with therapeutic CD47 blockade.

FIG. 5A-5B shows the result of tumor xenograft assays of NSG mice engrafted with HuT78 tumor cells, and treated with a PBS negative control or Magrolimab antibody; FIG. 5A tumor volume and FIG. 5B survival. Survival was improved, and tumor volume was reduced by the treatment with therapeutic CD47 blockade.

FIG. 6 provides a flow cytometry staining analysis for CTCL cells.

### DETAILED DESCRIPTION OF THE EMBODIMENTS

Methods are provided for the targeted depletion of CTCL cancer cells in a subject, where the cancer cells are selectively ablated by phagocytosis of the living cells, following contacting with an agent that blocks CD47 signaling.

To facilitate an understanding of the invention, a number of terms are defined below.

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### Definitions

Biological sample. The term “sample” with respect to an individual encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

DNA samples, e.g. samples useful in genotyping, are readily obtained from any nucleated cells of an individual, e.g. hair follicles, cheek swabs, white blood cells, etc., as known in the art.

The term “biological sample” encompasses a clinical sample. The types of “biological samples” include, but are not limited to: tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA).

Anti-CD47 agent. As used herein, the term “anti-CD47 agent” or “CD47-blocking agent” refers to any agent that reduces the binding of CD47 (e.g., on a target cell) to SIRPα (e.g., on a phagocytic cell). Non-limiting examples of suitable anti-CD47 reagents include SIRPα polypeptides, e.g. high affinity SIRPα polypeptides; anti-SIRPα antibodies; soluble CD47 polypeptides; and anti-CD47 antibodies or antibody fragments; and conjugates thereof, e.g. soluble SIRPα polypeptides conjugated to an Fc region polypeptide. In some embodiments, a suitable anti-CD47 agent specifically binds CD47 to reduce the binding of CD47 to SIRPα.

In some embodiments, a suitable anti-CD47 agent, e.g., an anti-SIRPα antibody, a soluble CD47 polypeptide, etc., specifically binds to SIRPα to reduce the binding of CD47 to SIRPα. A suitable anti-CD47 agent that binds SIRPα does not activate SIRPα (e.g., in the SIRPα-expressing phagocytic cell). The efficacy of a suitable anti-CD47 agent can be assessed by assaying the agent (further described below). In an exemplary assay, target cells are incubated in the presence or absence of the candidate agent. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding. When CD47 is activated, a process akin to apoptosis (i.e., programmed cell death) may occur (Manna and Frazier (2004) Cancer Research, 64, 1026-1036). Thus, in some embodiments, the anti-CD47 agent does not directly induce cell death of a CD47-expressing cell.

SIRPα polypeptide. A SIRPα polypeptide comprises the portion of SIRPα that is sufficient to bind CD47 at a recognizable affinity, which portion normally lies between the signal sequence and the transmembrane domain, or a fragment thereof that retains the binding activity. A suitable SIRPα polypeptide reduces (e.g., blocks, prevents, etc.) the interaction between the native proteins SIRPα and CD47. The SIRPα reagent will usually comprise at least the dl domain of SIRPα. In some embodiments, a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

Included as a SIRPα polypeptide are high-affinity variants of SIRPα as known and used in the art, including without limitation CV1-hIgG4, which has the set of amino acid substitutions relative to wild-type SIRPα of V6I; V27I; I31F; E47V; K53R; E54Q; H56P; S66T; V92I and is fused to an Fc region. High affinity SIRPα reagents are described in international application PCT/US13/21937, which is hereby specifically incorporated by reference. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more. The high affinity SIRPα reagent will usually comprise at least the dl domain of SIRPα with modified amino acid residues to increase affinity. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, etc.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47 antibody that is an “anti-CD47 agent” can be referred to as a “CD47-blocking antibody”. A non-limiting example of a non-blocking antibody is anti-CD47 antibody 2D3, which binds to CD47, but does not reduce the interaction between CD47 and SIRPα. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4, described for example, by Liu et al. (2015) Pre-Clinical Development of a Humanized Anti-CD47 Antibody with Anti-Cancer Therapeutic Potential. PLoS ONE 10(9): e0137345) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-SIRPα antibodies. Antibodies that specifically bind to human SIRPα are known and used in the art, and may be adapted by the use of an engineered Fc region. Exemplary antibodies include those described in international patent application WO 2015/138600; in published US application 2014/0242095 (University Health Networks); published application CN103665165 (JIANGSU KUANGYA BIOLOGICAL MEDICAL SCIENCE & TECHNOLOGY; Zhao X W et al. Proc Natl Acad Sci USA 108:18342-7 (2011), each herein specifically incorporated by reference. An anti-SIRPα antibody may be pan-specific, i.e. binding to two or more different human SIRPα isoforms; or may be specific for one isoform. For example, the antibody 1.23 A described by Zhang et al., supra. is reported to be specific for the SIRPα1 variant, while the 12C4 antibody is pan-specific. Anti-SIRPα antibodies can also be specific for SIRPα and lack binding to SIRPβ and/or SIRPγ. Anti-SIRPα antibodies can be pan-specific with respect to SIRPβ and/or SIRPγ.

Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

In certain embodiments, soluble CD47 polypeptide comprises the extracellular domain of CD47, including the signal peptide, such that the extracellular portion of CD47 is typically 142 amino acids in length, and has the amino acid sequence set forth in, for example, the Genbank reference sequence for human CD47, including NP_942088 or NP_001768.1. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the soluble CD47 polypeptides are secreted; accordingly, a polynucleotide encoding a soluble CD47 polypeptide may include a nucleotide sequence encoding a signal peptide that is associated with a polypeptide that is normally secreted from a cell.

In other embodiments, the soluble CD47 polypeptide comprises an extracellular domain of CD47 that lacks the signal peptide (124 amino acids). As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a soluble CD47 polypeptide comprises a CD47 extracellular domain variant. Such a soluble CD47 polypeptide retains the capability to bind to SIRPα without stimulating SIRPα signaling. The CD47 extracellular domain variant may have an amino acid sequence that is at least 65%-75%, 75%-80%, 80-85%, 85%-90%, or 95%-99% identical (which includes any percent identity between any one of the described ranges) to a reference human CD47 sequence.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those suspected of having cancer, etc.).

Cutaneous T cell lymphomas (CTCL) are a heterogenous group of extranodal non-Hodgkin lymphomas, which, by definition, are largely confined to the skin at diagnosis. Greater than 75% of primary cutaneous lymphomas are T-cell derived, two-thirds of which may be classified as Mycosis fungoides (MF) or Sezary Syndrome (SS). The incidence of CTCL increases significantly with age, with a median age at diagnosis in the mid-50's and a four-fold increase in incidence appreciated in patients over 70.

Differences in T cell phenotypes are reflected in the difference in CTCL subtypes. The majority of normal skin-resident T cells are CD45RO⁺ memory T cells expressing the skin-homing addressin CLA, which binds E-selectin on postcapillary venules in the skin and is required for lymphoccaltrainyte rolling. Skin-resident T cells highly express the chemokine receptors CCR4, CCR6, and CCR10, among others, that are required for their migration into the skin. In contrast to central memory T cells (T_{CM}), which express CCR7 and L-selectin, effector memory T cells (T_{EM}) form a persistent population of tissue-resident cells capable of rapidly responding to antigenic rechallenge and comprise 80% of T cells residing in normal skin. The malignant T cells in patients with leukemic CTCL variants (SS) have been shown to express CCR7 and L-selectin, resembling T_{CM}, while the malignant clone in MF lesions resembled T_{EM}. This difference in the putative cell of origin between SS (T_{CM} derived) and MF (T_{EM} derived) is consistent with their distinct clinical behavior, as T_{CM} may be found in both the peripheral blood, lymph node, and skin and are long-lived cells resistant to apoptosis, while skin-resident T_{EM} cells fail to circulate in peripheral blood, remaining fixed within the skin.

Extrinsic factors present within the tumor microenvironment may contribute to the growth and survival of malignant T cells; supported by the observation that cytokine supplementation or the provision of T-cell costimulatory signals supports the growth of malignant T cells in vitro. Both gene-expression profiling and immunohistochemistry-based studies have shown an important contribution of non-malignant cells, including monocyte-derived lymphoma-associated macrophages, in the pathogenesis of both Hodgkin and non-Hodgkin lymphomas. Similarly, malignant T cells in the skin are frequently associated with dendritic cells and immunohistochemistry based studies have clearly demonstrated an abundance of both lymphoma-associated macrophages and dendritic cells, many of which may be actively recruited into the tumor microenvironment by tumor-derived chemokines.

In addition to the tumor microenvironment's role, widespread impairment of cellular immunity has long been appreciated in CTCL and contributes to the significant morbidity and mortality associated with infectious complications observed in CTCL. Both quantitative and qualitative defects in natural killer (NK) cell, dendritic cell, and T cell-mediated immunity are observed in CTCL. In addition, CTCL is associated with a significant loss of the T-cell repertoire. In patients with advanced-stage disease, and half of patients with limited-stage disease, a dramatic loss of TCR diversity has been observed.

Mycosis fungoides. The definitive diagnosis of MF, particularly patch/plaque stage disease, is challenging, as many of its clinical and pathologic features are nonspecific. A diagnosis of MF may be made on the basis of clinical and histopathologic features alone, but determination of T-cell clonality and assessment for the aberrant loss of T-cell antigen expression by immunohistochemical staining for CD2, CD3, CD5, and CD7 are useful ancillary studies in the diagnosis of MF (and SS). The malignant lymphocytes in MF/SS are usually CD3⁺CD4⁺ and CD8⁻, but frequently lose the expression of other pan-T-cell antigens. A significant population of cells lacking CD2, CD5, and/or CD7 expression, either within the entire lesion or the epidermis alone, is highly specific for MF in most reported series. Clinically, patch/plaque stage MF is frequently characterized by persistent and progressive lesions that develop in a “bathing suit” distribution and vary in size, shape, and color.

Sézary syndrome. Traditionally, SS is defined as a leukemic form of CTCL associated with erythroderma. As in other chronic lymphoproliferative disorders, the Sezary cell count is preferably expressed in absolute terms, with ≥1000 cells/μl classified as B2 disease in the current ISCL/EORTC TNMB staging classification. The histologic findings in the skin often resemble those observed in MF, with less prominent epidermotropism, while lymph node involvement is characterized by complete effacement of the nodal architecture by infiltrating Sezary cells. In SS, clonal T cells are generally CD3⁺CD4⁺ and CD8⁻. As in MF, the aberrant loss of pan-T-cell antigens, including CD2, CD3, CD4, CD5, and CD7 is frequently observed. Loss of CD26 expression is also useful in the identification of Sezary cells, being observed in the majority of cases. The currently proposed ISCL criteria for SS integrate clinical, histologic, immunophenotyping, and molecular studies. In patients with erythroderma, criteria recommended for the diagnosis of SS by the ISCL include the following: absolute Sezary count ≥1000 cells/μ1, a CD4/CD8 ratio ≥10 (due to the clonal expansion of CD4⁺ cells), aberrant expression of pan-T-cell antigens, demonstration of T-cell clonality by Southern blot or PCR-based methods, or cytogenetic demonstration of an abnormal clone.

TNMB (tumor, node, metastasis, and blood) staging remains an important prognostic factor in MF/SS and forms the basis for a “risk-adapted” approach to treatment. Patients with only patches and plaques have Stage I disease, but may be further divided into Stage IA (<10% body surface area involved or T1) or Stage IB (>10% body surface area involved or T2) based on the extent of skin involvement. Patients with patch/plaque stage disease (T1/T2) and architectural preservation of any clinically abnormal lymph nodes are classified as Stage IIA. Collectively, patients with Stage I and IIA disease have “limited-stage” disease, as the overall survival in these patients is measured in decades, with survival in patients with Stage IA disease resembling that of normal age-matched controls. In contrast, patients with tumor stage disease (T3), erythroderma (T4), nodal involvement characterized by partial or complete architectural effacement (N3), visceral metastases (M1), or significant leukemic involvement (B2) have “advanced-stage” disease. Detection of a clonal TCR gene rearrangement is an adverse prognostic factor.

CTCL patients presenting with patch/plaque stage MF (limited stage) generally have a good prognosis, and may be treated with expectant management or skin directed therapies. Such patients may benefit from treatment with anti-CD47 agents, including combinations with skin directed therapy.

Patients with advanced-stage MF/SS benefit from treatment with anti-CD47 agents, which may be combined with additional skin-directed therapies, biologic-response modifiers, and sequential use of systemic chemotherapeutic agents.

Combination therapies. Treatment with an anti-CD47 agent can be combined with additional therapies. Included among therapies currently approved for CTCL are retinoids, e.g. the oral RXR-selective “rexinoid” bexarotene and a topical gel formulation, e.g. at a dose of 300 mg/m². Other combinations include hypomethylating agents, and BCL inhibitors.

A number of HDAC inhibitors are used in cancer treatment and may find use in combination therapies with an anti-CD47 agent. Vorinostat (suberoylanilide hydroxamic acid, SAHA) and romidepsin (depsipeptide) inhibit class I and II HDACs (i.e., pan-HDAC inhibitors) and are used in the treatment of CTCL. Additional HDAC inhibitors that may find use include, without limitation: Chidamide, Panobinostat, Belinostat, Panobinostat, Valproic acid, Mocetinostat, Abexinostat, Entinostat, SB939, Resminostat, Givinostat, Quisinostat, HBI-8000, Kevetrin, CUDC-101, AR-42, CHR-2845, CHR-3996, 4SC-202, CG200745, ACY-1215, ME-344, and sulforaphane.

Interferon-alpha has pleiotropic effects in CTCL. Interferons have been reported to interact with CD47 signaling pathways, and can be used in combination with anti-CD47 treatment.

Other therapeutic modalities frequently used in the management of these patients, including PUVA, bexarotene, chemotherapy, and extracorporeal photophoresis (ECP). During ECP pooled leukapheresis and plasmapheresis products are exposed to 8-methoxypsoralen (8-MOP) prior to extracorporeal circulation through a 1 mm thick disposable cassette exposed to UVA radiation. The irradiated leukocytes are subsequently reinfused. ECP leads to monocyte activation, including significant changes in gene expression, and dendritic cell differentiation, which is thought to culminate in enhanced antigen presentation and the initiation of a host immune response. A modified ECP protocol (i.e., “transimmunization”) whereby blood products are incubated overnight following UVA irradiation and prior to patient infusion has also been developed. ECP is FDA approved for the treatment of CTCL and is the treatment of choice in the first-line management of many patients with Sezary syndrome in many centers. ECP is generally performed for 2 consecutive days every 2-4 weeks. Benefits can be obtained by combining ECP and monocyte activation with anti-CD47 treatment, which increases phagocytosis mediated by innate immune system cells.

In some embodiments, anti-CD47 therapy is combined with administration of targeted therapeutics that include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax, venetoclax, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

In some embodiments, anti-CD47 therapy is combined with administration of hypomethylating (also known as epigenetic) agents for combination with an anti-CD47 agent. A hypomethylating agent is a drug that inhibits DNA methylation. Currently available hypomethylating agents block the activity of DNA methyltransferase (DNA methyltransferase inhibitors/DNMT inhibitors). Currently two members of the class, azacitidine and decitabine are FDA-approved for use in the United States. Guadecitabine is also of interest. Because of their relatively mild side effects, azacitidine and decitabine are particularly feasible for the treatment of older patients and patients with co-morbidities. Dosing of azacitidine or decitabine may be conventional, e.g. from about 50-100 mg/m² daily dose, and may be around 75 mg/m²/daily.

In other embodiments anti-CD47 therapy is combined with administration of Bcl-2 inhibitors, such as obatoclax, venetoclax, and gossypol. Dosing of, for example, venetoclax, may be conventional, e.g. an escalating oral dose starting at 20 mg/daily and increasing to up to 400 mg/daily, as tolerated by the patient. An exemplary dosing schedule is provided in Example 3. In certain embodiments a triple combination is administered, of CD47 blockade, hypomethylating agent, and Bcl-2 inhibitor.

Anti-tumor antibodies. Antibodies useful in combination with an anti-CD47 agent include antibodies that bind to an epitope present, usually selectively present, on the CTCL cells, which include without limitation CD52, CCR4, CD30, etc. Preferred antibodies also comprise an active Fc sequence that binds to an Fc receptor present on macrophages. Antibodies may be administered systemically or subcutaneously.

Alemtuzumab is a humanized IgG1 monoclonal antibody directed against CD52, an antigen widely expressed by B-cells, T-cells, and monocytes, which can be combined with anti-CD47 therapy. Given the risk of infectious complications, low-dose subcutaneous alemtuzumab has been used to treat SS, e.g. 3 mg of subcutaneous alemtuzumab on day 1 followed by a 10 mg dose on alternating days until the Sezary count was <1000 mm³.

Monoclonal antibodies targeting additional T-cell specific antigens, including CD2, CD4, CD25 and CCR4 may be used for this purpose. Mogamulizumab (KW-0761) is a humanized monoclonal antibody specific for the chemokine receptor CCR4 that has been defucosylated and is consequently associated with enhanced antibody-dependent cell-mediated cytotoxicity. Brentuximab vedotin is an antibody-drug conjugate in which an anti-CD30 monoclonal antibody is linked with an anti-tubulin agent (monomethyl auristatin E).

Tumor targeted antibodies can be administered at a dose of from about 0.05 mg/kg, 0.1 mg/kg; 0.5 mg/kg, 1 mg/kg, 2.5 mg/kg, 5 mg/kg, 7.5 mg/kg, 10 mg/kg, 12.5 mg/kg, 15 mg/kg, or more as required. Dosing may be daily, every other day, semi-weekly, weekly, every 14 days, etc. for a period of time sufficient to achieve the desired result, e.g. from about 1, 2, 3, 4, 5, 6, 7, 8, 9, 10, 11, 12 or more weeks. In some embodiments the dose for a combination therapy is lower than the dose required for effectiveness as a monotherapy. In some embodiments the T cell targeted antibody is administered sub-cutaneously.

Systemic chemotherapy. Systemic chemotherapy in CTCL is generally reserved for patients with advanced-stage MF/SS who have either relapsed following therapy with skin-directed therapies and the biologic-response modifiers described above or have extensive disease with visceral organ involvement. While combination chemotherapy regimens (e.g., CHOP) are associated with response rates exceeding 70-80%, the responses achieved are frequently short-lived and are associated with significant myelosuppression and infectious complications. Low-doses of oral chemotherapy, including methotrexate, cyclophosphamide, chlorambucil, or etoposide, may be considered. For patients with an adequate performance status, single-agent gemcitabine, pegylated liposomal doxorubicin, and pentostatin have been used. Pegylated liposomal doxorubicin is generally well tolerated, with a lower incidence of neutropenia than gemcitabine, but with occasional infusion related and mucocutaneous toxicities, including palmoplantar erythrodysesthesia. Pentostatin is associated with fewer complete responses and significant lymphopenia-associated immunosuppression. Pralatrexate is a novel antifolate with a high affinity for the reduced folate carrier (RFC-1) and novel mechanism of resistance. In an effort to reduce the incidence of mucositis, folic acid and vitamin B12 supplementation is routinely provided in these patients. Additional agents, including bortezomib, are being explored.

Unfortunately, the duration of response with these agents is frequently measured in months. Therefore, novel therapeutic agents, such as those provided herein, alone or in combination, are needed.

As used herein, “antibody” includes reference to an immunoglobulin molecule immunologically reactive with a particular antigen, and includes both polyclonal and monoclonal antibodies, e.g. an entire tetrameric IgG protein. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)2, Fab, Fv and rIgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin, particularly in IgG4 Fc region.

Antibodies of interest may be tested for their ability to induce ADCC (antibody-dependent cellular cytotoxicity) or ADCP (antibody dependent cellular phagocytosis). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.).

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a cancer therapeutic drug, ESA or tumor-directed antibody with a pharmaceutical composition of the present invention means administration with the CD47 reagent at such time that both the drug, ESA or antibody and the composition of the present invention will have a therapeutic effect. Such concomitant administration may involve concurrent (i.e. at the same time), prior, or subsequent administration of the drug, ESA or antibody with respect to the administration of a compound of the invention. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., 01-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

A “therapeutically effective dose” or “therapeutic dose” is an amount sufficient to effect desired clinical results (i.e., achieve therapeutic efficacy). For purposes of this invention, a therapeutically effective dose of an anti-CD47 agent is an amount that is sufficient to palliate, ameliorate, stabilize, reverse, prevent, slow or delay the progression of the disease state by increasing phagocytosis of a target cell (e.g., a target cell); for example to reduce the number of tumor cells in the blood, bone marrow, etc. Thus, a therapeutically effective dose of an anti-CD47 agent reduces the binding of CD47 on an target cell, to SIRPα on a phagocytic cell, at an effective dose for increasing the phagocytosis of the target cell.

As an indicator for a therapeutically effective dose, which takes into account the complex interplay between antigen sink, biological activities of the agent and requirement for enhancing phagocytosis of cancer cells, the therapeutic dose can be determined as equivalent to a dose that provides for substantially complete occupancy of CD47 binding sites on the surface of cancer cells by an anti-CD47 agent (referred to herein as receptor occupancy), for a defined period of time. In some embodiments the anti-CD47 agent specifically binds to CD47. Substantially complete receptor occupancy may be at least about 75%, at least about 80%, at least about 85%, at least about 90%, at least about 95%, at least about 98%, or more.

As a surrogate for receptor occupancy, the serum levels of the anti-CD47 agent can be determined. In some embodiments, a therapeutically effective dose leads to sustained serum levels of anti-CD47 agent (e.g., an anti-CD47 antibody) of about 40 μg/ml or more (e.g., about 50 ug/ml or more, about 60 ug/ml or more, about 75 ug/ml or more, about 100 μg/ml or more, about 125 ug/ml or more, or about 150 ug/ml or more). In some embodiments, a therapeutically effective dose leads to sustained serum levels of anti-CD47 agent (e.g., an anti-CD47 antibody) that range from about 40 μg/ml to about 300 μg/ml, up to about 500 μg/ml, up to about 750 μg/ml, up to about 1000 μg/ml, e.g, from about 40 μg/ml to about 1000 μg/ml, from about 40 μg/ml to about 800 μg/ml, from about 40 μg/ml to about 700 μg/ml, from about 40 μg/ml to about 600 μg/ml, from about 50 μg/ml to about 500 μg/ml, from about 50 μg/ml to about 750 μg/ml, from about 50 μg/ml to about 300 μg/ml, from about 50 μg/ml to about 250 μg/ml, from about 75 μg/ml to about 1000 μg/ml from about 75 μg/ml to about 750 μg/ml, from about 75 μg/ml to about 500 μg/ml, from about 75 μg/ml to about 250 μg/ml, from about 100 μg/ml to about 1000 μg/ml, from about 100 μg/ml to about 600 μg/ml, or from about 100 μg/ml to about 300 μg/ml). In some embodiments, a therapeutically effective dose for treating hematologic malignancies leads to sustained serum levels of anti-CD47 agent (e.g., an anti-CD47 antibody) of about 50 μg/ml or more (e.g., sustained serum levels of 75 μg/ml or more; or sustained serum levels that range from about 50 μg/ml to about 150 μg/ml).

A therapeutically effective dose or a series of therapeutically effective doses can achieve and maintain a serum level of anti-CD47 agent, and/or substantially complete receptor occupancy. A therapeutically effective dose of an anti-CD47 agent can depend on the specific agent used, but is usually about 8 mg/kg body weight or more (e.g., about 8 mg/kg or more, about 10 mg/kg or more, about 15 mg/kg or more, about 20 mg/kg or more, about 25 mg/kg or more, about 30 mg/kg or more, about 35 mg/kg or more, or about 40 mg/kg or more), or about 45 mg/kg or more, or about 50 mg/kg, or about 60 mg/kg or more. Ranges may include from about 10 mg/kg to about 60 mg/kg (e.g., from about 10 mg/kg to about 50 mg/kg, or from about 10 mg/kg to about 30 mg/kg). The dose required to achieve and/or maintain a particular therapeutic dose is proportional to the amount of time between doses and inversely proportional to the number of doses administered. Thus, as the frequency of dosing increases, the required dose decreases. The optimization of dosing strategies will be readily understood and practiced by one of ordinary skill in the art.

### Methods of Use

Methods are provided for treating or reducing cutaneous T cell lymphomas (CTCL), in a regimen comprising contacting the targeted cells with an agents that that blockades CD47 activity. Such methods include administering to a subject in need of treatment a therapeutically effective amount of the agent of the invention, including without limitation combinations of the reagent with a chemotherapeutic drug, radiation therapy, or an ESA.

Effective doses of the agent of the present invention for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

Chemotherapeutic agents that can be administered in combination with an anti-CD47 agent include, without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination with an anti-CD47 agent may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax, venetoclax, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

An anti-CD47 agent may be administered in combination with an immunomodulator, such as a cytokine, a lymphokine, a monokine, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TGF-α or TGF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and LT.

In some embodiments the additional therapeutic entity in an immune response modulator. Immune checkpoint proteins are immune inhibitory molecules that act to decrease immune responsiveness toward a target cell, particularly against a tumor cell in the methods of the invention. Endogenous responses to tumors by T cells can be dysregulated by tumor cells activating immune checkpoints (immune inhibitory proteins) and inhibiting co-stimulatory receptors (immune activating proteins). The class of therapeutic agents referred to in the art as “immune checkpoint inhibitors” reverses the inhibition of immune responses through administering antagonists of inhibitory signals. Other immunotherapies administer agonists of immune costimulatory molecules to increase responsiveness.

The immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors. The clinical activity of antibodies that block either of these receptors implies that antitumor immunity can be enhanced at multiple levels and that combinatorial strategies can be intelligently designed, guided by mechanistic considerations and preclinical models.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity. CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation.

Other immune-checkpoint proteins are PD1 and PDL1. Antibodies in current clinical use against these targets include nivolumab and pembrolizumab. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand. Because many tumors are highly infiltrated with T_{Reg} cells, blockade of the PD1 pathway may also enhance antitumor immune responses by diminishing the number and/or suppressive activity of intratumoral T_{Reg} cells.

Lymphocyte activation gene 3 (LAGS; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy. Antibody targeting of these receptors can be used in the methods of the invention.

TIM3 inhibits T helper 1 (TH1) cell responses, and TIM3 antibodies enhance antitumor immunity. TIM3 has also been reported to be co-expressed with PD1 on tumor-specific CD8⁺ T cells. Tim3 blocking agents can overcome this inhibitory signaling and maintain or restore anti-tumor T cell function.

BTLA is an inhibitory receptor on T cells that interacts with TNFRSF14. BTLA^{hi} T cells are inhibited in the presence of its ligand. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions. Dual blockade of BTLA and PD1 enhances antitumor immunity.

A2aR, the ligand of which is adenosine, inhibits T cell responses, in part by driving CD4+ T cells to express FOXP3 and hence to develop into T_{Reg} cells. Deletion of this receptor results in enhanced and sometimes pathological inflammatory responses to infection. A2aR can be inhibited either by antibodies that block adenosine binding or by adenosine analogues.

Agents that agonize an immune costimulatory molecule are also useful in the methods of the invention. Such agents include agonists or CD40 and OX40. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L). Stimulation through CD40 activates macrophages. One of the major effects of CD47 blocking agents is to enhance phagocytosis of target cells by macrophages and other phagocytes. Therefore, combining agonistic CD40 ligands with anti CD47 can enhance the therapeutic efficacy compared to each mono therapy (example 1). Agonistic CD40 agents may be administered substantially simultaneously with anti-CD47 agents; or may be administered prior to and concurrently with treatment with anti-CD47 to pre-activate macrophages.

Agents that alter the immune tumor microenvironment are useful in the methods of the invention. Such agents include IDO inhibitors which inhibit the production of indoleamine-2,3-dioxygenase (IDO), an enzyme that exhibits an immunosuppressive effect.

Other immuno-oncology agents that can be administered in combination with CD47 blockade according to the methods described herein include antibodies specific for chemokine receptors, including without limitation anti-CCR4 and anti-CCR2. Anti CCR4 (CD194) antibodies of interest include humanized monoclonal antibodies directed against C-C chemokine receptor 4 (CCR4) with potential anti-inflammatory and antineoplastic activities. Exemplary is mogamulizumab, which selectively binds to and blocks the activity of CCR4, which may inhibit CCR4-mediated signal transduction pathways and, so, chemokine-mediated cellular migration and proliferation of T cells, and chemokine-mediated angiogenesis. In addition, this agent may induce antibody-dependent cell-mediated cytotoxicity (ADCC) against CCR4-positive T cells. CCR4, a G-coupled-protein receptor for C-C chemokines such MIP-1, RANTES, TARC and MCP-1, is expressed on the surfaces of some types of T cells, endothelial cells, and some types of neurons. CCR4, also known as CD194, may be overexpressed on adult T-cell lymphoma (ATL) and peripheral T-cell lymphoma (PTCL) cells.

The combination therapy described above may be combined with other agents that act on regulatory T cells, e.g. anti-CTLA4 Ab, or other T cell checkpoint inhibitors, e.g. anti-PD1, anti-PDL1 antibodies, and the like.

In some embodiments, administration of a combination of agents of the invention is combined with an effective dose of an agent that increases patient hematocrit, for example erythropoietin stimulating agents (ESA). Such agents are known and used in the art, including, for example, Aranesp® (darbepoetin alfa), Epogen®/Procrit® (epoetin alfa), Omontys® (peginesatide), Procrit®, etc. See, for example, U.S. Pat. No. 9,623,079.

In some embodiments, the therapeutic dosage of the anti-CD47 agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor growth, tumor metastasis or tumor invasion of CTCL. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Compositions for the treatment of cancer can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the compositions (e.g. anti-CD47 agents, and formulations thereof) of the invention and instructions for use. The kit can further contain a least one additional reagent, e.g. a chemotherapeutic drug, ESA, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially ablate targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### EXPERIMENTAL

### Example 1

### Treatment of Cutaneous T Cell Lymphoma with CD47 Blockade; and Combined with Anti-CCR4

Cancer Cells. HH (CTCL lymphoblast ATCC); HuT78 (Sezary Syndrome CTCL, ATCC); HuT102 (Mycosis Fungoides CTCL, ATCC); and MJ (Mycosis Fungoides CTCL, ATCC) were cultured in RPMI (ThermoFisher S.) (DLD1), EMEM (ThermoFisher S.) (CACO-2, LS174T), McCoy's 5A (ThermoFisher S.) (HT29, HCT116), or Leibovitz's L-15 (ThermoFisher S.) (SW48, SW 620) supplemented with 10% fetal bovine serum (Omega Scientific), 100 U/mL penicillin and 100 μg/mL streptomycin (ThermoFisher S.)

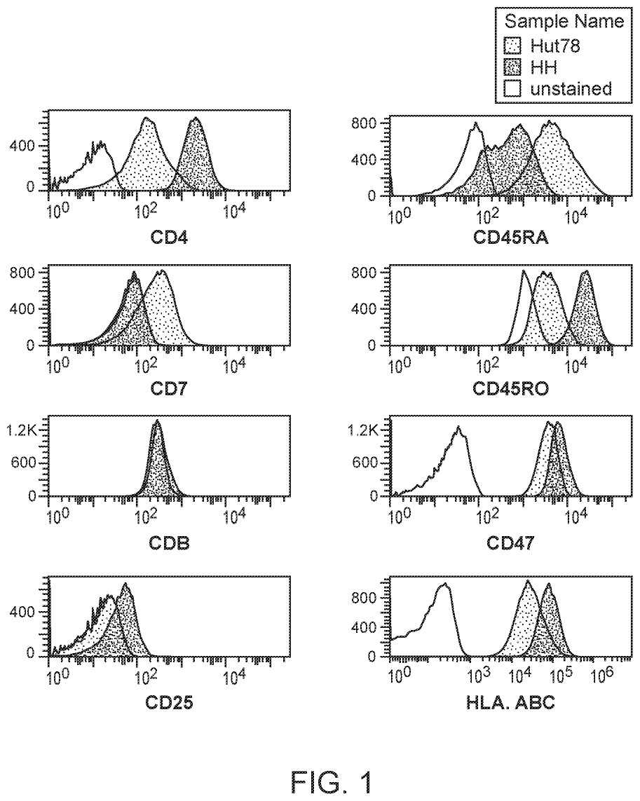

Cell line surface marker profiling. Cultured cells were combined with antibodies specific for the markers as referenced, and the level of staining monitored by flow cytometry. Results are shown in FIG. 1. It is shown that both HH and Hut78 are positive for expression of CD4, CD45RAA, CD45RO, and HLA. Both cells are negative for expression of CD8 and CD25; and HH is negative for CD7, while Hut78 is positive.

Mice. 6-8 week old NSG (NOD.Cg-Prkdcscid Il2rgtm1Wjl/SzJ) mice were used. The Stanford colony of these mice was founded by mice purchased from Jackson Labs (Stock 005557).

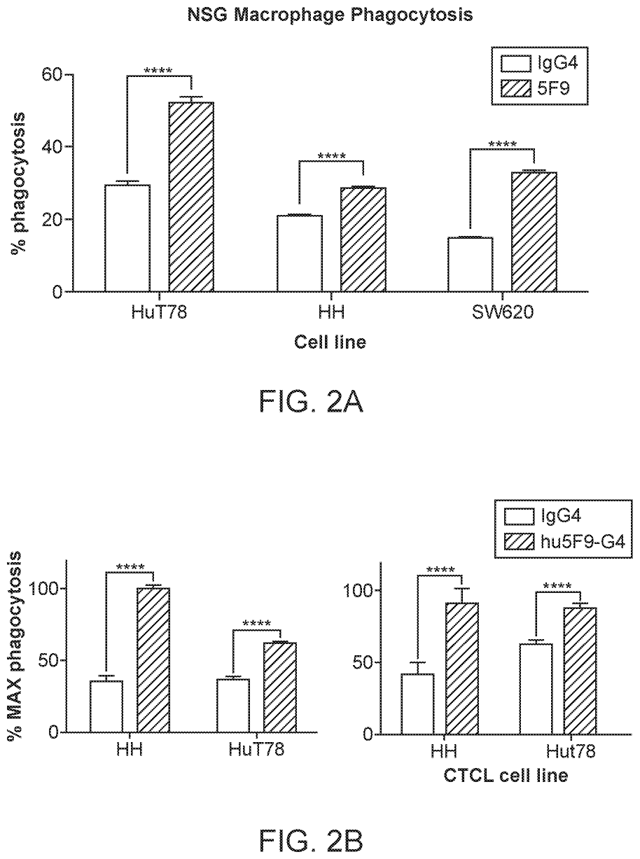

In Vitro Phagocytosis Assay. Peripheral blood mononuclear cells were enriched by density gradient centrifugation and monocytes were purified with anti-CD14 microbeads (Miltenyi) and differentiated to macrophages by culture for 7-10 days in IMDM+GlutaMax (Invitrogen) supplemented with 10% AB-Human Serum (Invitrogen) and 100 U/mL penicillin and 100 μg/mL streptomycin (Invitrogen). Phagocytosis assays were performed by co-culture of 50,000 macrophages with 100,000 CTCL tumor cells for 2 hours, then analyzed using an LSRFortessa cell analyzer with high throughput sampler (BD Biosciences). Antibodies used for treatment included: IgG4 isotype control, anti-CD47—clone magrolimab (Stanford). Macrophages were identified by flow cytometry using anti-CD206 antibody. Dead cells were excluded from the analysis by staining with DAPI (Sigma). Phagocytosis was evaluated as the percentage of GFP+ macrophages and was normalized to the maximal response by each independent donor against each cell line. Data is shown in FIGS. 2A-2B. It can be seen that there is a significant increase in phagocytosis when cells are incubated with 5F9 and both mouse (FIG. 2A) and human (FIG. 2B) macrophages.

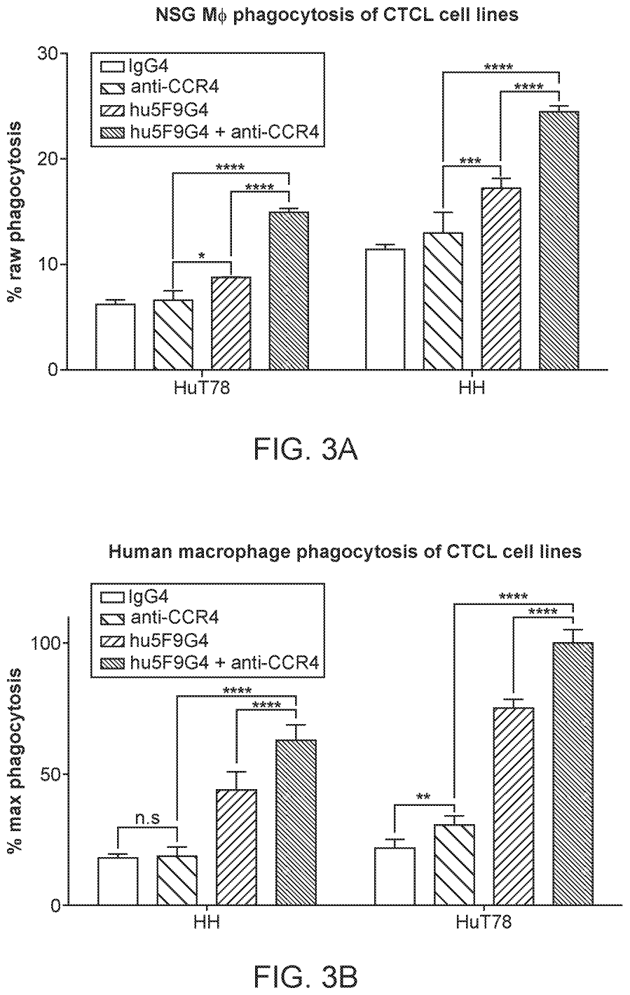

The phagocytosis experiment was also performed using a combination of magrolimab and anti-CCR4, data shown in FIGS. 3A-3B, showing a significant increase in phagocytosis with the combined therapy.

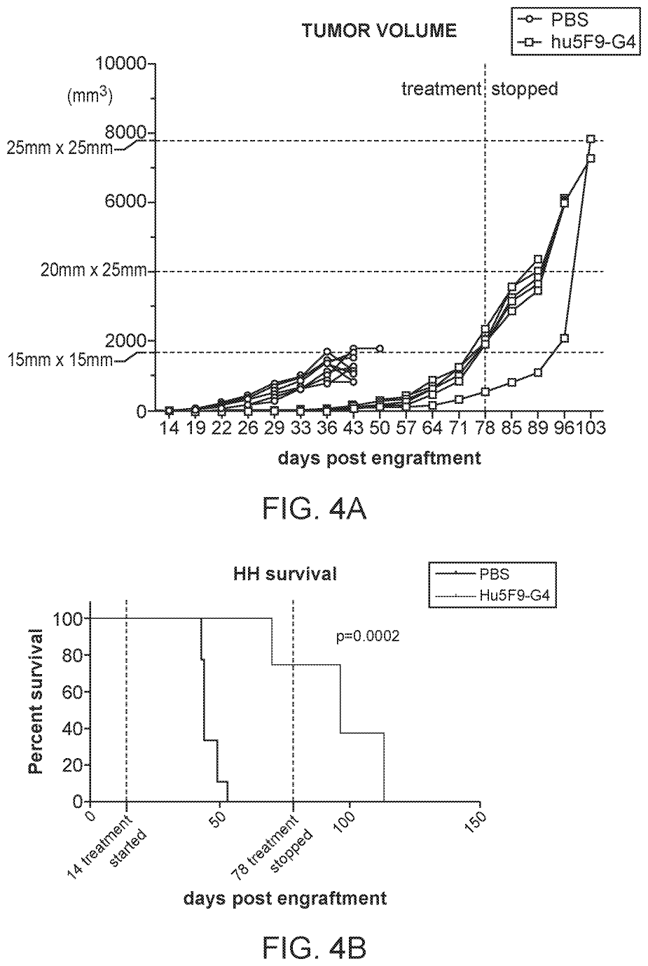

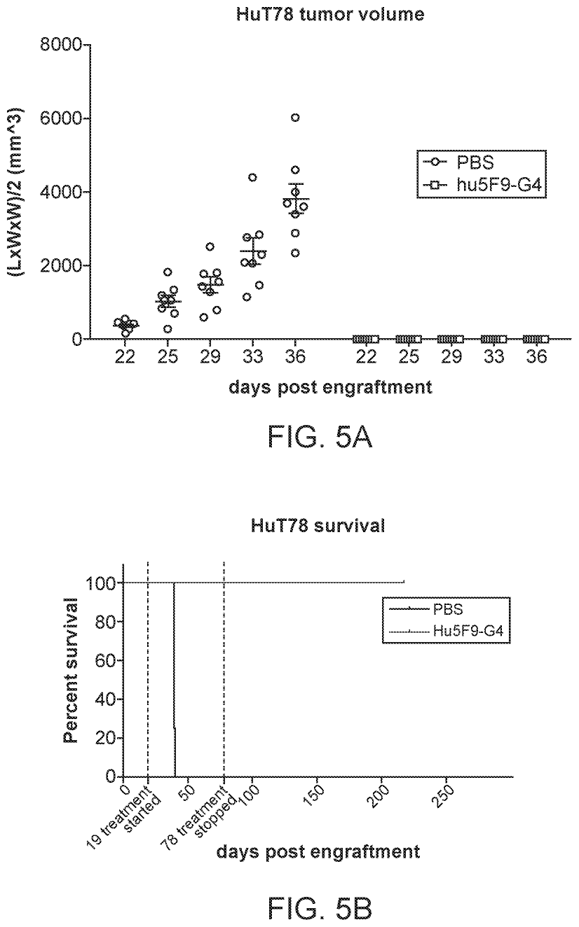

Xenograft Tumor Models. 125,000 HH cells or HuT cells were transplanted with 50% Matrigel (BD) onto the flank of NSG mice. In both models, treatment was initiated upon confirmation of tumor engraftment at day 14 or 19, and continued as indicated. For all treatments, antibodies where administered by intraperitoneal injection (100 μl) as follows: PBS and Humagrolimab (250 μg) every other day. Tumor volume and survival was monitored. The data is shown in FIGS. 4A-B (HH) and FIGS. 5A-B (Hut78).

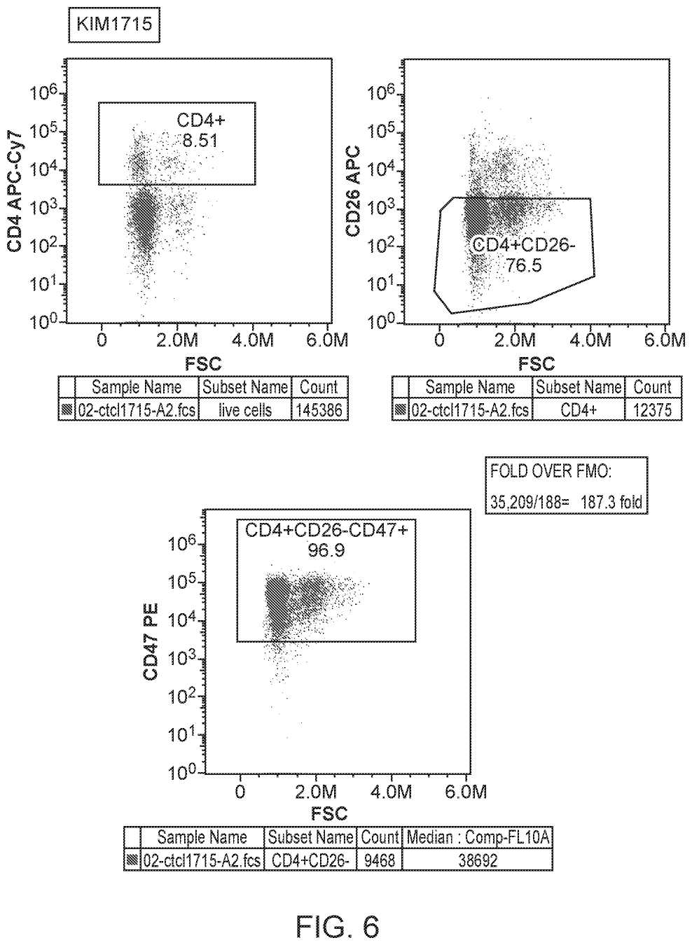

Human patient samples were screened for expression of CD47. As exemplified in the data shown in FIG. 6, the blast cell population in the human blood sample, when gated for the population of CD4⁺CD26⁻ cells were found to express high levels of CD47. There was a greater than 6-fold increase in CD47 expression for CD4⁺ cells v. CD4⁻ cells.

### Example 2

### A Study of Azacitidine Plus Magrolimab Anti-CD47 Antibody in Patients with Cutaneous T Cell Lymphoma

This trial will evaluate magrolimab, a monoclonal antibody designed to block CD47, in combination with azacitidine for treatment of patients with CTCL. Azacitidine is a drug currently used for treatment of AML or MDS in patients who are not eligible for typical chemotherapy. Cutaneous T cell lymphoma (CTCL) is an incurable skin-homing T cell non-Hodgkin lymphoma (NHL) that may commonly have blood/leukemic and lymph node involvement. Advanced stages of CTCL are more likely to have blood/leukemic and lymph node involvement and show resistance to standard therapy.

The major aims of the study are: to evaluate the efficacy of magrolimab in combination with azacitidine in previously untreated CTCL; and to evaluate the efficacy in relapsed/refractory CTCL, as measured by the objective response rate.

Objective Response—Number of Participants with Objective Response ie, confirmed complete or partial response according to RECIST Version 1.1). Time to Tumor Response (TTR) is defined for patients with confirmed objective response (CR or PR) as the time from the date of randomization or date of first dose of study treatment to the first documentation of objective tumor response. Duration of Response (DR) is defined for patients with confirmed objective response (CR or PR) as the time from the first documentation of objective tumor response to the first documentation of objective tumor progression or to death due to any cause, whichever occurs first. Progression-Free Survival (PFS) is defined as the time from the date of randomization or date of first dose of study treatment to the date of disease progression by RECIST v1.1 or death due to any cause, whichever occurs first. Overall Survival (OS) is defined as the time from the date of randomization or date of first dose of study treatment to the date of death.

Azacitidine is a nucleoside analog, specifically a chemical analog of cytidine. Azacitidine has two known primary anti-neoplastic mechanisms of action: 1) inhibition of deoxyribonucleic acid (DNA) methyltransferase leading to hypomethylation of DNA and 2) direct cytotoxicity of malignant hematopoietic cells through cell death via its incorporation into DNA and ribonucleic acid (RNA). As a hypomethylating agent, azacitidine is a standard-of-care therapy for newly diagnosed AML patients who are ineligible for induction chemotherapy or HSCT based on age, co-morbidities, or other factors. Azacitidine is also SOC and approved in the United States (US) for treatment of subtypes of MDS. In a large international randomized Phase 3 study comparing azacitidine to conventional care regimens in newly diagnosed AML patients 65 years or older, azacitidine was administered at 75 mg/m² per day subcutaneously (SC) for 7 consecutive days per 28-day treatment cycle for at least 6 cycles. Median OS for azacitidine was 10.4 months, which was statistically significant improvement over conventional care regimens (6.5 months).

The AE profile of azacitidine primarily includes myelosuppression (anemia, leukopenia, and thrombocytopenia) and clinical sequelae of myelosuppression (neutropenic fever, infections, bleeding, and fatigue). Common non-hematologic AEs include gastrointestinal events (diarrhea, nausea, constipation, and decreased appetite), skin disorders (rash, pruritus, petechiae, ecchymosis), injection site/infusion-related reactions (injection site erythema and/or pain for SC administration) and fever. Generally these toxicities can be managed with supportive care interventions, pharmacologic treatment, or dose delays and/or adjustments.

| Arms | Assigned Interventions |
|---|---|
| Experimental: | Drug: Azacitidine |
| Azacitidine + | 75 mg/m² per day subcutaneously (SC) for 7 consecutive |
| magrolimab | days per 28-day treatment cycle for at least 6 cycles |
| | Drug: magrolimab |
| | following a priming dose of 1 mg/kg, magrolimab is |
| | dosed at increasing dose levels ranging from 1-100 |
| | mg/kg, IV |

Adverse Events [Time Frame: 28 days]
Adverse events according to National Cancer Institute Common Terminology Criteria for Adverse Events (NCI CTCAE) Version 4.03 or customized AE severity grading as defined in the protocol.

### Example 3

### A Study of Venetoclax Plus Magrolimab Anti-CD47 Antibody in Patients with Cutaneous T Cell Lymphoma

This trial will evaluate magrolimab, a monoclonal antibody designed to block CD47, in combination with venetoclax for treatment of patients with CTCL. Cutaneous T cell lymphoma (CTCL) is an incurable skin-homing T cell non-Hodgkin lymphoma (NHL) that may commonly have blood/leukemic and lymph node involvement. Advanced stages of CTCL are more likely to have blood/leukemic and lymph node involvement and show resistance to standard therapy.

The major aims of the study are: to evaluate the efficacy of magrolimab in combination with venetoclax in previously untreated CTCL; and to evaluate the efficacy in relapsed/refractory CTCL, as measured by the objective response rate. The combined treatment is alternatively administered in combination with one or more of azacitidine, decitabine, or low-dose cytarabine.

Objective Response—Number of Participants with Objective Response ie, confirmed complete or partial response according to RECIST Version 1.1). Time to Tumor Response (TTR) is defined for patients with confirmed objective response (CR or PR) as the time from the date of randomization or date of first dose of study treatment to the first documentation of objective tumor response. Duration of Response (DR) is defined for patients with confirmed objective response (CR or PR) as the time from the first documentation of objective tumor response to the first documentation of objective tumor progression or to death due to any cause, whichever occurs first. Progression-Free Survival (PFS) is defined as the time from the date of randomization or date of first dose of study treatment to the date of disease progression by RECIST v1.1 or death due to any cause, whichever occurs first. Overall Survival (OS) is defined as the time from the date of randomization or date of first dose of study treatment to the date of death.

Venetoclax is a newly approved oral agent for the treatment of CLL. Venetoclax has also been shown to be especially potent against NHL and AML cell lines expressing high levels of Bcl-2. Malignant cells isolated from a cohort of CTCL patients have been shown to exhibit extreme sensitivity to venetoclax by cell viability assays, with EC₅₀ doses as low as 1-3 nM, and this sensitivity correlated with Bcl-2 expression. Thus, venetoclax is a promising therapy for CTCL. The ramp up schedule for Venetoclax may be as follows:

Daily Dose Venetoclax

| | |
|---|---|
| Week 1 | 20 mg |
| Week 2 | 50 mg |
| Week 3 | 100 mg |
| Week 4 | 200 mg |
| Week 5 and beyond | 400 mg |

| Arms | Assigned Interventions |
|---|---|
| Experimental: | Drug: Venetoclax |
| Venetoclax + | Eligible patients will be enrolled into the study and |
| magrolimab | receive venetoclax daily per the US FDA package insert |
| | guidelines of venetoclax, with dose escalation up to |
| | 400 mg. To minimize the risk of tumor lysis syndrome |
| | (TLS), and following the package insert directions for |
| | dose escalation over 5 weeks, the initial dose is 20 mg |
| | daily, and may be progressively increased as tolerated |
| | to 400 mg by week 5. |
| | Drug: magrolimab |
| | following a priming dose of 1 mg/kg, magrolimab is |
| | dosed at increasing dose levels ranging from 1-100 |
| | mg/kg, IV |
| Experimental: | Drug: Venetoclax |
| venetoclax + | Eligible patients will be enrolled into the study and |
| magrolimab + | receive venetoclax daily per the US FDA package insert |
| azacitidine | guidelines of venetoclax, with dose escalation up to |
| | 400 mg. To minimize the risk of tumor lysis syndrome |
| | (TLS), and following the package insert directions for |
| | dose escalation over 5 weeks, the initial dose is 20 mg |
| | daily, and may be progressively increased as tolerated |
| | to 400 mg by week 5. |
| | Drug: Azacitidine |
| | 75 mg/m² per day subcutaneously (SC) for 7 consecutive |
| | days per 28-day treatment cycle for at least 6 cycles |
| | Drug: magrolimab |
| | following a priming dose of 1 mg/kg, magrolimab is |
| | dosed at increasing dose levels ranging from 1-100 |
| | mg/kg, IV |

Adverse Events [Time Frame: 28 days]. Adverse events according to National Cancer Institute Common Terminology Criteria for Adverse Events (NCI CTCAE) Version 4.03 or customized AE severity grading as defined in the protocol.

Exclusion Factors
Extracutaneous disease except blood, bone marrow and lymph nodes.
Concomitant use of any systemic anti-cancer therapy or immune modifier.
Concomitant use of moderate or strong CYP3A inhibitors or inducers within 1 week of initiation of study drug administration.
Patients with biopsy confirmed transformed MF.
Prior allogeneic hematopoietic cell transplant.
Any ongoing infection requiring antibiotics within 2 weeks prior to the start of the study drug, except for antibiotics (e.g. cephalexin) prescribed superficial skin infection.
Known history of human immunodeficiency virus (HIV), hepatitis B or C.
History of prior malignancy with the exception of cervical intraepithelial neoplasia, non-melanoma skin cancer, and adequately treated localized prostate carcinoma (PSA <1.0). Patients with a history of other malignancies must have undergone potentially curative therapy and have no evidence of that disease for five years.
Uncontrolled intercurrent illness, condition, or circumstances that could limit compliance with the study including, but not limited to, the following: acute or chronic graft versus host disease, uncontrolled diabetes mellitus or hypertension, or psychiatric conditions.
Major surgery within 8 weeks of enrollment.
Cerebrovascular event (transient ischemic attack, stroke or CNS bleeding) within the last 12 months.
Major bleeding within the last 6 months.
Use of any investigational agents within 30 days prior to enrollment and for the duration of the study
Pregnant or lactating
Unwilling or unable to provide informed consent
   Medically significant cardiac event or unstable cardiovascular function defined as:
Symptomatic ischemia, unstable angina pectoris
Uncontrolled clinically significant cardiac arrhythmia
Symptomatic heart failure NYHA Class ≥3
Myocardial infarction or cardiac surgery within 6 months prior to enrollment

Primary Outcome Measures:

Body Temperature [Time Frame: Up to 32 weeks] Safety and tolerability endpoints will be evaluated on the basis of body temperature.

Blood Pressure—Diastolic [Time Frame: Up to 32 weeks]. Safety and tolerability endpoints will be evaluated on the basis of blood pressure.

Blood Pressure—Systolic [Time Frame: Up to 32 weeks]. Safety and tolerability endpoints will be evaluated on the basis of blood pressure.

Pulse Rate [Time Frame: Up to 32 weeks]. Safety and tolerability endpoints will be evaluated on the basis of pulse rate.

Respiratory Rate [Time Frame: Up to 32 weeks]. Safety and tolerability endpoints will be evaluated on the basis of respiratory rate.

Adverse Events [Time Frame: Up to 32 weeks]. Adverse events will be used to measure the study defined outcome:Toxicity. Toxicity (as adverse events) will measured according to the NCI CTCAE (v5.0) for AEs and clinical laboratory profile; AEs will be collected in all patients who received at least one dose of venetoclax and up to four weeks after last dose (Termination visit).

Secondary Outcome Measures:

Skin Clinical Response [Time Frame: Up to 32 weeks]. Exploratory skin clinical responses measured by a modified severity-weighted assessment tool (mSWAT).

Duration of Response [Time Frame: Up to 32 weeks]. Duration of response to treatment will be measured in weeks.

Relapse Free and Progression Free Survival [Time Frame: Up to 32 weeks]. Relapse free and progression free survival based on every 4 week follow up after the initial dose until one of the events occurs first: Progressive disease (PD) is documented, another anticancer treatment is administered and/or 28 weeks are completed after the patient's first dose of venetoclax.

What is claimed is:

1. A method of targeting cutaneous T cell lymphoma for immunodepletion in a subject, the method comprising:
contacting a population of cells comprising the targeted cells with an agent that blockades CD47 activity; in a dose effective to increase depletion of the targeted cells.

2. The method of claim 1, wherein the contacting is performed in the presence of phagocytic cells.

3. The method of claim 2, wherein the contacting is performed on an individual human in vivo.

4. The method of claim 3, wherein the treatment provides for increased overall survival of the individual.

5. The method of any of claims 1-4, wherein the agent that agent that blockades CD47 activity is an anti-CD47 antibody.

6. The method of claim 5, wherein the anti-CD47 antibody comprises an IgG4 Fc region.

7. The method of claim 6, wherein the antibody is magrolimab.

8. The method of claim 5, further comprising administration of a priming dose of the agent that blockades CD47 activity.

9. The method according to any of claims 1-8, wherein the method further comprises treating the subject with a one or more additional anti-cancer treatments.

10. The method of claim 9, wherein the additional anti-cancer treatment is administered concomitantly with the agent that blockades CD47 activity.

11. The method of claim 9 or claim 10, wherein the additional anti-cancer treatment is selected from a skin-directed therapy; a biologic-response modifier; a tumor-targeted antibody; chemotherapy; and extracorporeal photophoresis.

12. The method of claim 11, wherein the additional anti-cancer treatment is administration of an effective dose of a retinoid.

13. The method of claim 11, wherein the additional anti-cancer treatment is administration of an effective dose of an HDAC inhibitor.

14. The method of claim 11, wherein the additional anti-cancer treatment is administration of an effective dose of interferon alpha.

15. The method of claim 11, wherein the additional anti-cancer treatment is administration of an effective dose of an HDAC inhibitor.

16. The method of claim 11, wherein the additional anti-cancer treatment is administration of an effective dose of a Bcl-2 inhibitor.

17. The method of claim 15, wherein the additional anti-cancer treatment is administration of an effective dose of venetoclax.

18. The method of claim 15, further comprising administration of an effective dose of a hypomethylating agent.

19. The method of claim 11 wherein the additional anti-cancer treatment is administration of an effective dose of a hypomethylating agent.

20. The method of claim 18, wherein the hypomethylating agent is azacitidine or gemcitabine.
